(19) United States
(12) Patent Application Publication
(10) Pub. No.: US 2012/0151634 A1
(43) Pub. Date: Jun. 14, 2012

(54) ASSAY FOR MEASURING ROOTWORM RESISTANCE

(75) Inventor: Aaron John Gassmann, Ames, IA (US)

(73) Assignee: IOWA STATE UNIVERSITY RESEARCH FOUNDATION, INC., Ames, IA (US)

(21) Appl. No.: 13/314,375

(22) Filed: Dec. 8, 2011

Related U.S. Application Data
(60) Provisional application No. 61/459,465, filed on Dec. 13, 2010.

(51) Int. Cl. A01H 5/00

(52) U.S. Cl. 800/302; 800/320.1; 800/320; 800/322; 800/312; 800/320.3; 800/320.2; 800/314; 800/306; 800/317.2; 800/317.4

(57) ABSTRACT

A method for determining the resistance of a pest to a plant that produces insecticidal toxins. The method involves sampling pests in fields with known pest problems, and in control fields with no known pest problems. Eggs are obtained from the sample pest populations, and larvae hatched from the eggs are evaluated in laboratory bioassays for their survival on two transgenic crop hybrids, each of which contain a unique toxin targeting the pest, and two near isogenic hybrids that lack a gene for the toxin. Mortality rates are determined by counting the larvae recovered, and measuring the larvae instars based on head capsule width to calculate the resistance levels.

### CROSS-REFERENCE TO RELATED APPLICATIONS

This application claims priority under 35 U.S.C. §119 to provisional application Ser. No. 61/459,465 filed Dec. 13, 2010, herein incorporated by reference in its entirety.

### FIELD OF THE INVENTION

The present invention relates to methods for measuring the development of resistant pests to transgenic plants.

### BACKGROUND OF THE INVENTION

Insects, nematodes, and related arthropods annually destroy an estimated 15% of agricultural crops in the United States and even more than that in developing countries. Yearly, these pests cause over $100 billion dollars in crop damage in the U.S. alone. Corn rootworm (CRW) can result in yield losses of 8-16% of the total 11.8 million bushels of grain harvested (2004).

Some of this damage occurs in the soil when plant pathogens, insects and other such soil borne pests attack the seed after planting. In the production of corn, for example, much of the damage is caused by rootworms, insect pests that feed upon or otherwise damage the plant roots, and by cutworms, European corn borers, and other pests that feed upon or damage the above ground parts of the plant. General descriptions of the type and mechanisms of attack of pests on agricultural crops are provided by, e.g., Metcalf (1962), in Destructive and Useful Insects, 4th ed. (McGraw-Hill Book Co., NY); and Agrios (1988), in Plant Pathology, 3d ed. (Academic Press, NY).

In an ongoing seasonal battle, farmers must apply billions of gallons of synthetic pesticides to combat these pests. However, synthetic pesticides pose many problems. They are expensive, costing U.S. farmers alone almost $8 billion dollars per year. The use of pesticides results in the selection of individuals resistant to the pesticide, and can lead to the development of pesticide-resistant populations. Resistance to chemical insecticides such as organochlorines, organophosphates, carbamates, spinosyns and pyrethroids is known.

Because of concern about the impact of pesticides on public health and the health of the environment, significant efforts have been made to find ways to reduce the amount of chemical pesticides that are used. Recently, much of this effort has focused on the development of transgenic crops that are engineered to express insect toxicants derived from microorganisms. For example, the soil bacterium Bacillus thuringiensis (“Bt”) contains genes encoding insecticidal proteins. Pesticidal crystal proteins derived from Bt are commonly referred to as “Cry proteins” or “Cry peptides.” The Cry proteins are globular protein molecules which accumulate as protoxins in crystalline form during late stage of the sporulation of Bt. After ingestion by the pest, the crystals are solubilized to release protoxins in the alkaline midgut environment of the larvae. Protoxins (˜130 kDa) are converted into toxic fragments (˜66 kDa N terminal region) by gut proteases. The protoxin attacks the insect midgut, stops feeding and eventually kills susceptible insects. Gill et al., Annu Rev. Entomol. 37:615 (1992); Fischhoff, In Biotechnology and Integrated Pest Management, Ed. G J Persley, pp. 214-227, CAB International, Cambridge, UK. While many of these proteins are quite toxic to specific target insects, nevertheless, they are harmless to plants and other non-targeted organisms. See Lambert and Peferoen, BioScience, 42:112 (1992); Gill et al., Annu Rev. Entomol. 37:615 (1992); Meadows, In: Bacillus thuringiensis, An Environmental Biopesticide: Theory and Practice, Entwistle et al., Eds., pp. 193-200 (1993). Some Cry proteins have been recombinantly expressed in crop plants to provide pest-resistant transgenic plants. Among those, Bt-transgenic cotton and corn have been widely cultivated. For instance, transgenic crops engineered to produce insecticidal toxins derived from Bt were planted on more than 58 million hectares worldwide in 2010.

One concern in planting transgenic pest resistant plants is that resistant insect pests will emerge. Laboratory and field evidence documents that many pests are capable of evolving high levels of resistance to a number of commonly used Bt toxins. Tabashnik, Annu Rev. Entomol. 39:47 (1994); Tabashnik, J. Econ. Entomol. 83:1671 (1990); Bauer, Fla. Ent. 78:414 (1995); Gould, Proc. Natl. Acad Sci. USA 94:3519 (1997). Resistance may evolve whether the Bt is applied to plants or the plants are genetically engineered to express Bt. The development of resistance to Bt toxin-expressing crops may also result in resistance to commercial formulations of fermented strains of Bt, such as DIPEL.®. (Abbott Laboratories). Consequently, resistance based on the expression of a single gene might eventually be lost due to the evolution of Bt resistance in the insects.

Because of the foregoing concerns, rapid, reliable methods to distinguish Bt-susceptible from Bt-resistant species, and to detect the development of Bt resistance, as well as resistance to other insecticides, in populations of insects, are desirable. Traditional methods involve placing Bt toxin on the surface of an artificial diet for a larval test subject, typically Diabrotica virgifera virgifera. These methods are problematic in that Bt toxin is present only on the surface of the artificial diet, thus, the insect may chew through the toxin and then be unaffected for the remainder of the assay. The results of these assay are further skewed in that mold is a persistent problem when using an artificial diet and it does not provide for complete larval development. Thus, it is important and necessary that novel, accurate methods be developed in order to insure successful management of the pest and to promote durability of a resistance trait in maize and other plants. One can see that there is a continuing need in the art for simple, effective, quick ways to determine continued pest resistance in maize or other plants to pests.

### BRIEF SUMMARY OF THE INVENTION

The invention discloses methods for determining the resistance of a pest to a pest resistant plant. Pests include, but are not limited to, insects selected from the orders Coleoptera, Diptera, Hymenoptera, Lepidoptera, Mallophaga, Homoptera, Hemiptera, Orthoptera, Thysanoptera, Dermaptera, Isoptera, Anoplura, Siphonaptera, and Trichoptera. In one embodiment of the invention, the level of resistance of a pest is quantified by a novel assay method. The first step of this method involves obtaining a sample of pests from a field with pests suspected of being resistant to pest resistant plants. The sample pests are allowed to reproduce, and their eggs are cultivated until larvae are hatched. The larvae are next evaluated in for their survival on pest resistant plants and non-pest resistance plants grown in a greenhouse under controlled watering, lighting and fertilizing conditions. After growing for a set time, the plants are moved to incubators for the bioassays. Larvae are then placed upon exposed roots of each pest resistant plant. After a period of incubation, the aboveground biomass of the plants are excised and the soil, containing the roots and larvae, are removed and placed on a funnel to extract larvae from the soil. Mortality rates are then calculated by counting the larvae recovered, and measuring the larvae instars based on head capsule width to determine the resistance levels of the pest to the pest resistant plants. These results are compared with the mortality rates of pests to non pest resistant plants conducted in similar bioassays.

### DESCRIPTION OF FIGURES

FIGS. 1-9 represent an example assay with steps to measure rootworm resistance to transgenic maize.

FIGS. 1A, 1B. Sample rootworm populations from the field are placed into cages and fed a controlled diet of corn leaf tissue and an artificial western corn rootworm diet.

FIG. 2. Eggs are obtained from the sample rootworm populations are placed in a cold room for at least 5 months, stored, washed, and placed atop moistened sieved soil held in a 10 cm Petri dish.

FIG. 3. Maize seeds used in the bioassays were initially treated with CruiserMaxx 250 containing the neonicotinoid insecticide Thiamethoxam. This seed treatment was removed prior to planting by a washing process. The seeds are first washed using a dish detergent solution. The seeds are washed for a total of one hour broken up into three 20-minute sessions. Each session washes the seed in approximately 250-300 mL of DI water with 2-3 drops of dish detergent. The solution is agitated using a magnetic stir bar. After the final session, the seed is rinsed in DI water approximately 5 times and laid out to dry for approximately 12 hours (overnight). The seed is then washed using a 10% bleach solution. The seed sits in the bleach solution for one hour stirred with a spoon every 15 minutes. After the hour, the seed is rinsed in DI water 10 times to remove any possible leftover bleach. The seed is then laid out to dry for no less than 24 hours.

FIG. 4. Plants are grown in the greenhouse for two-four weeks. They receive Scott's Excel 15-5-15 NPK+Calcium and Magnesium weekly at the standard rate. A 50:50 mix of Sungro LC1 and SB300 potting soils produced by Sunshine was utilized but other types of potting soil is also appropriate.

FIG. 5. Newly hatched larvae are placed on the exposed roots of the plants.

FIG. 6. Plants are trimmed to approximately 20 cm (leaving some leaf tissue) and placed in a grow chamber at 25 degrees Celsius; 16/8/L/D; 65% RH. This specific height allows for placement into an environmental chamber. After approximately seventeen days under these conditions the rootworms will develop to the late stage of the final larval instar but will not pupate.

FIGS. 7A, 7B, 7C. Larvae from the assay cups are extracted using a Berlese funnel.

FIG. 8. The number of larvae are counted and their instar determined based on head capsule width. This is accomplished using a digital camera and grid. The grid is developed for the specific purpose of measuring larval instars. The grid is divided into rectangles with the short side calibrated to 280 nm and the long side calibrated 420 nm. These dimensions allow for discrimination between the 1st, 2nd and 3rd instars.

FIG. 9. Data is compiled to measure susceptibility of a population. The data below are from a laboratory colony, which is highly susceptible to transgenic corn. Bar heights are sample means and error bars are the standard error of the mean. Instars are the developmental stages of the rootworm larvae, which hatch from eggs as first instars and then form a pupa after completing the third instar. On the x-axis, Isoline is corn that does not contain rootworm active Bt, VT Triple has one type of rootworm active Bt (Cry3Bb1) and SmartStax has two types of rootworm active Bt (Cry3Bb1 and Cry34/Cry35). Note that as the number of toxins increases rootworm development is slowed and survival decreases.

### DETAILED DESCRIPTION OF THE INVENTION

In the description that follows, a number of terms are used extensively. The following definitions are provided to facilitate understanding of the embodiments of the invention.

The article “a” and “an” are used herein to refer to one or more than one (i.e., to at least one) of the grammatical object of the article. By way of example, “an element” means one or more element. As used herein, the term “comprising” means “including but not limited to.”

As used herein, the terms “corn” or “maize” includes all plant varieties that can be bred with Zea mays, corn and maize, including wild maize species. In one embodiment, the disclosed methods are useful for monitoring resistance in a plot of pest resistant corn, where corn is systematically followed by corn (i.e., continuous corn). In another embodiment, the methods are useful for monitoring resistance in a plot of first-year pest resistant corn, that is, where corn is followed by another crop (e.g., soybeans), in a two-year rotation cycle. Other rotation cycles are also contemplated in the context of the invention, for example where corn is followed by multiple years of one or more other crops, so as to prevent resistance in other extended diapause pests that may develop over time.

As used herein, the term “creating or enhancing insect resistance” is intended to mean that the plant, which has been genetically modified in accordance with the methods of the present invention, has increased resistance to one or more insect pests relative to a plant having a similar genetic component with the exception of the genetic modification described herein.

By “crop plants” is intended a plant, purposely planted and harvested or utilized. Preferably, crop plants are monocotyledonous or dicotyledonous plants and are used for such purposes as, but not limited to, food, feed, fuel and combinations thereof. Crop plants include, but are not limited to, maize, sorghum, sunflower, soybean, wheat, alfalfa, rice, cotton, canola, barley, millet, potato and tomato. A particularly preferred monocotyledonous crop plant is maize.

As used herein “pest” and “pests” include, but are not limited to, insects selected from the orders Coleoptera, Diptera, Hymenoptera, Lepidoptera, Mallophaga, Homoptera, Hemiptera, Orthoptera, Thysanoptera, Dermaptera, Isoptera, Anoplura, Siphonaptera, and Trichoptera.

As used herein, the term “pesticidal” is used to refer to a negative effect, such as but not limited to a toxic effect against a pest (e.g., CRW), and includes activity of either, or both, an externally supplied pesticide and/or an agent that is produced by the crop plants.

As used herein, the terms “pesticidal activity” and “insecticidal activity” are used synonymously to refer to activity of an organism or a substance (such as, for example, a protein) that can be measured, by way of non-limiting example, via pest mortality, retardation of pest development, pest weight loss, pest repellency, and other behavioral and physical changes of a pest after feeding and exposure for an appropriate length of time. In this manner, pesticidal activity often impacts at least one measurable parameter of pest fitness. For example, the pesticide or pesticidal property may be a polypeptide to decrease or inhibit insect feeding and/or to increase insect mortality upon ingestion of the polypeptide limit the amount of damage that the pest can cause to a root mass. Assays for assessing pesticidal activity are well known in the art. See, e.g., U.S. Pat. Nos. 6,570,005 and 6,339,144.

As used herein, the term “pesticidal gene” or “pesticidal polynucleotide” refers to a nucleotide sequence that encodes a polypeptide that exhibits pesticidal activity. As used herein, the terms “pesticidal polypeptide,” “pesticidal protein,” or “toxin” are intended to mean a protein having pesticidal activity.

As used herein, the term “pesticidally effective amount” connotes a quantity of a substance or organism that has pesticidal activity when present in the environment of a pest. For each substance or organism, the pesticidally effective amount is determined empirically for each pest affected in a specific environment. Similarly an “insecticidally effective amount” may be used to refer to a “pesticidally effective amount” when the pest is an insect pest.

As used herein, the term “plant” includes reference to whole plants, plant organs (e.g., leaves, stems, roots, etc.), seeds, plant cells, plant protoplasts, plant cell tissue cultures from which plants can be regenerated, plant calli, plant clumps, and plant cells that are intact in plants or parts of plants and progeny of same. Parts of transgenic plants are to be understood within the scope of the invention to comprise, for example, plant cells, protoplasts, tissues, callus, embryos as well as flowers, pollen, ovules, seeds, branches, kernels, ears, cobs, husks, stalks, stems, fruits, leaves, roots, root tips, anthers, and the like, originating in transgenic plants or their progeny previously transformed with a DNA molecule of the invention and therefore consisting at least in part of transgenic cells, are also an object of the present invention. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides.

As used herein, the term “plant cell” includes, without limitation, seeds, suspension cultures, embryos, meristematic regions, callus tissue, leaves, roots, shoots, gametophytes, sporophytes, pollen, and microspores. The class of plants that can be used in the methods of the invention is generally as broad as the class of higher plants amenable to transformation techniques, including both monocotyledonous and dicotyledonous plants.

A “field” is intended to mean an area where crops are planted, comprising one or more fields or areas of indeterminate size.

A “problem field” includes fields identified by farmers as those with a known pest problem and a high abundance of pests, such as “severe feeding injury” to Bt maize by corn rootworm displaying plants that are goosenecked (bent at the plant-soil interface) and lodged (tilted in a pronounced manner), which are characteristic of severe feeding injury by corn rootworm larvae.

A “control field” includes fields not associated with unexpected problems by pests.

As used herein, the term “polypeptide,” “peptide,” and “protein” are used interchangeably herein to refer to a polymer of amino acid residues. The terms apply to amino acid polymers in which one or more amino acid residues is an artificial chemical analogue of a corresponding naturally-occurring amino acid, as well as to naturally-occurring amino acid polymers.

As used herein, a “pest resistant plant” is intended to mean a plant that limits or eliminates insect pest-related damage to a plant by, for example, inhibiting the ability of the insect pest to grow, feed, and/or reproduce or by killing the insect pest. The phrase includes, but is not limited to, deterring the insect pest from feeding further on the plant, harming the insect pest by, for example, inhibiting the ability of the insect to grow, feed, and/or reproduce, or killing the insect pest. A “non pest resistant plant” is a plant that does not have such capabilities to limit or eliminate insect pest-related damage.

A “trait,” as used herein, refers to the phenotype derived from a particular sequence or groups of sequences. A single expression cassette may contain both a nucleotide encoding a pesticidal protein of interest, and at least one additional gene, such as a gene employed to increase or improve a desired quality of the transgenic plant. Alternatively, the additional gene(s) can be provided on multiple expression cassettes. The combinations generated can also include multiple copies of any one of the polynucleotides of interest. Additionally, either a single expression cassette or multiple expression cassettes may encode pesticidal activity or other activities or markers which may be useful in determining trait expression.

As used herein, the term “transgenic” includes any cell, cell line, callus, tissue, plant part, or plant, the genotype of which has been altered by the presence of heterologous nucleic acid including those transgenics initially so altered as well as those created by sexual crosses or asexual propagation from the initial transgenic. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

As used herein, the term “transgenic pest resistant plant” means a plant or progeny thereof (including seeds) derived from a transformed plant cell or protoplast, wherein the plant DNA contains an introduced heterologous DNA molecule, not originally present in a native, non-transgenic plant of the same strain, that confers resistance to one or more pests. In a preferred embodiment these pests are rootworms, such as but not limited to, corn rootworms. In another embodiment, these pests are WCRW, NCRW and MCRW. The term refers to the original transformant and progeny of the transformant that include the heterologous DNA. The term also refers to progeny produced by a sexual outcross between the transformant and another variety that includes the heterologous DNA. It is also to be understood that two different transgenic plants can also be mated to produce offspring that contain two or more independently segregating, added, heterologous genes. Selfing of appropriate progeny can produce plants that are homozygous for both added, heterologous genes. Back-crossing to a parental plant and out-crossing with a non-transgenic plant are also contemplated, as is vegetative propagation. Descriptions of other breeding methods that are commonly used for different traits and crop plants can be found in one of several references, e.g., Fehr (1987), in Breeding Methods for Cultivar Development, ed. J. Wilcox (American Society of Agronomy, Madison, Wis.).

As used herein, “soil” can include all types of soil, although a 1:1 ratio of Sunshine Sun Gro SB300 and Sunshine Sun Gro LC1 potting soils (Sun Gro Horticulture Canada

Ltd., Vancouver, British Columbia) was used in the present invention.

A “Berlese funnel” as used in the disclosure was invented by Antonio Berlese (subsequently modified by Albert Tullgren and is sometimes known as a Tullgren funnel), and is a device for extracting soil insects and other micro fauna from leaf litter. The funnel incorporates a funnel containing the soil or litter and a heat source, such as an electric lamp, that heats the litter. Animals escape from the desiccation of the litter by descending through a filter into a preservative liquid in a receptacle.

“Instar” as used herein, is a developmental stage of arthropods, such as insects, between each molt (ecdysis), until sexual maturity is reached. For most insect species the term “instar” is used to denote the developmental stage of the larval or nymphal forms of holometabolous (complete metamorphism) or hemimetabolous (incomplete metamorphism) insects, but the term can be used to describe any developmental stage including pupa or imago (the adult, which does not molt in insects).

The present invention utilizes an improved method for determining resistance of a pest to pest resistant plants. This improvement allows for complete development of the pest, uses an assay free from mold, and incorporates a diet for the pest as it would be found in naturally occurring transgenic plant tissue in the field. This method can be used to screen field populations for their level of resistance to current transgenic technologies. The method disclosed herein solves the problems of the prior art, by allowing for complete development of the insect, uses an assay free from mold, and incorporates a diet for the insect as it would be found naturally occurring in pesticidal transgenic plant tissue in the field. This method can be used to screen field populations for their level of resistance to current transgenic technologies. It can also be readily used by academics and private companies that monitor pest populations for resistance, and can also be used to test pest populations against newly developed transgenics.

In an exemplary embodiment of this invention, the level of resistance of the western corn rootworm to Bt maize is quantified by a novel assay method. According to the invention, a representative sample of western corn rootworm populations are obtained from problem fields and control fields. Problem fields are identified as fields with severe feeding injury to Bt maize by the western corn rootworm. Control fields are not associated with unexpected feeding by the western corn rootworm on Bt maize. Sample western corn rootworm populations collected from both problem and control fields are caged and fed a controlled diet. Eggs obtained from these populations are stored, washed, and placed atop moistened sieved soil. Neonate larvae hatched from the eggs are evaluated in laboratory bioassays for their survival on maize which contain a unique Bt toxin targeting corn rootworm, and two near isogenic maize hybrids that lack a gene for a rootworm active Bt toxin. Maize plants used in the bioassays are grown in separate containers, fertilized weekly, and grown in a greenhouse with supplemental lighting. After growing for approximately three to four weeks, the plants are moved to incubators for the bioassays. Larvae are placed at the base of a maize plant on an exposed root, and distributed equally between Bt and non-Bt maize plants. After a period of incubation, the aboveground biomass of the plant is excised and the soil containing the roots and larvae, are removed and placed on a Berlese funnel to extract larvae from the soil. Mortality rates are calculated by counting the larvae recovered, and measuring the larvae instars based on head capsule width to determine the resistance levels to Bt maize.

In another embodiment of the invention the method can be applied to different types of pests, such as larvae and adults of the order Coleoptera including cucumber beetles, rootworms, leaf beetles, potato beetles, and leafminers in the family Chrysomelidae (including, but not limited to: Diabrotica virgifera virgifera LeConte (western corn rootworm); D. barberi Smith & Lawrence (northern corn rootworm); D. undecimpunctata howardi Barber (southern corn rootworm); Colaspis brunnea Fabricius (grape colaspis); chafers and other beetles from the family Scarabaeidae (including, but not limited to: Popillia japonica Newman (Japanese beetle); Cyclocephala borealis Arrow (northern masked chafer, white grub); C. immaculata Olivier (southern masked chafer, white grub); Rhizotrogus majalis Razoumowsky (European chafer); Phyllophaga crinita Burmeister (white grub); and wireworms from the family Elateridae, Eleodes spp., Melanotus spp.; Conoderus spp.; Limonius spp.; Agriotes spp.; Ctenicera spp.; Aeolus spp. Pests can also include other invertebrates, such as nematodes, and plant diseases of plant roots, such as, for example, diseases of bacterial, fungal, or other origin that affect root health.

Since 2003, transgenic CRW-protected Bacillus thuringiensis (Bt) corn has been available to farmers (Monsanto Cry3Bb1) targeting the western corn rootworm (WCRW, Diabrotica virgifera virgifera, LeConte), northern corn rootworm (NCRW, D. barberi, Smith and Lawrence) and Mexican corn rootworm (MCRW, D. virgifera zeae, Krysan and Smith). WCRW is the most prevalent rootworm target pest in the United States with NCRW second and the MCRW is limited to Texas. Cry 34 and Cry35 are effective against WCRW, NCRW and MCRW. In the lifecycle of a CRW an adult female deposits eggs in a corn field during late summer; eggs overwinter and hatch in late-spring (late May-early June); larvae feed on corn roots for 3-4 weeks; mature into adult beetles which emerge from the soil in mid-July to feed on corn plants; mate and deposit eggs. Recently, deviations from the traditional lifestyle have occurred. One biotype of WCRW that is depositing its eggs in soybeans (and possibly other crop habitats) is now capable of causing significant injury to first-year corn (i.e., corn that has not systematically followed corn). This biotype is commonly called first-year corn rootworm or rotation-resistant corn rootworm. Another deviation has been seen in NCRW, especially in the northwestern region of the Corn Belt, where first-year corn may also be susceptible to rootworm injury when eggs remain in the soil for more than a year. In this situation, the eggs deposited in the plot remain dormant throughout the following year and then hatch the next year, when corn may again be planted in a two-year rotation cycle. Such rootworm activity is called extended diapause. Both of these deviations are examples of adaptations which lessen the effectiveness of crop rotation in pest management thus increasing the demand for other methods such as transgenic crops.

Also, U.S. Pat. No. 5,877,012 to Estruch et al. discloses the cloning and expression of proteins from such organisms as Bacillus, Pseudomonas, Clavibacter and Rhizobium into plants to obtain transgenic plants with resistance to such pests as black cutworms, armyworms, several borers and other insect pests. Publication WO/EP97/07089 by Privalle et al. teaches the transformation of monocotyledons, such as corn, with a recombinant DNA sequence encoding peroxidase for the protection of the plant from feeding by corn borers, earworms and cutworms. Jansens et al. (1997) Crop Sci., 37(5): 1616-1624, reported the production of transgenic corn containing a gene encoding a crystalline protein from Bt that controlled both generations of European Corn Borer (ECB). U.S. Pat. Nos. 5,625,136 and 5,859,336 to Koziel et al. reported that the transformation of corn with a gene from Bt that encoded for a δ-endotoxin provided the transgenic corn with improved resistance to ECB. A comprehensive report of field trials of transgenic corn that expresses an insecticidal protein from Bacillus thuringiensis (Bt) has been provided by Armstrong et al., in Crop Science, 35(2):550-557 (1995).

In an additional embodiment of the invention the method can be applied to various pest resistant plants. Transgenic crop plants expressing the Bt gene are widely cultivated. For instance, plants transformed to carry the Bt gene and express insecticidal proteins are known in the art, and include potato, cotton, tomato, corn, tobacco, lettuce and canola. Krimsky and Wrubel, Agricultural Biotechnology: An Environmental Outlook, Tufts University, Department of Urban and Environmental Policy, p. 29 (1993). See also U.S. Pat. No. 5,608,142; U.S. Pat. No. 5,495,071; U.S. Pat. No. 5,349,124; and U.S. Pat. No. 5,254,799. The use of such genetically engineered plants is expected to reduce the use of broad spectrum insecticides. Gasser and Fraley, Science 244:1293 (1989).

Various embodiments of the invention can further utilize different types of resistance to pests. Several hundred strains of Bacillus thuringiensis exist, with considerable specificity toward various groups of insects such as the lepidoptera (butterflies and moths), coleoptera (beetles) and/or diptera (mosquitoes), as well as toward nematodes. There is a species specificity of the interaction between Bt toxin and the membranes of insect gut cells. The Bt toxin of a particular B. thuringiensis strain may bind to the gut of lepidopteran larvae, or only some species of lepidopteran larvae, but not to others. Binding of the protein to the membrane is required for its toxic effects. Formulations of Bt toxin for use as insecticides are known in the art. See, e.g., U.S. Pat. No. 5,747,450; U.S. Pat. No. 5,250,515; U.S. Pat. No. 5,024,837; U.S. Pat. No. 4,797,276; and U.S. Pat. No. 4,713,241. A large number of Cry proteins have been isolated, characterized and classified based on amino acid sequence homology (Crickmore et al., 1998, Microbiol. Mol. Biol. Rev., 62: 807-813). This classification scheme provides a systematic mechanism for naming and categorizing newly discovered Cry proteins. The Cry1 classification is the best known and contains the highest number of cry genes which currently totals over 130. Specific, non-limiting examples of Bt Cry toxins of interest include the group consisting of Cry 1 (such as Cry1A, Cry1A(a), Cry1A(b), Cry1A(c), Cry1C, Cry1D, Cry1E, Cry1F), Cry 2 (such as Cry2A), Cry 3 (such as Cry3Bb), Cry 5, Cry 8 (see GenBank Accession Nos. CAD57542, CAD57543, see also U.S. patent application Ser. No. 10/746,914), Cry 9 (such as Cry9C) and Cry34/35, as well as functional fragments, chimeric or shuffled modifications, or other variants thereof. These insect toxins include, but are not limited to, the Cry toxins, including, for example, Cry1, Cry3, Cry5, Cry8, Cry9, Cry 34, and Cry 35.

In certain applications the plants produce more than one pesticidal activity, for example, via gene stacking For example, DNA constructs in the plants of the embodiments may comprise any combination of stacked nucleotide sequences of interest in order to create plants with a desired trait. Alternatively, plants having different pesticidal activities may be planted in the same plot. For example, a mixture of transgenic seed may contain different modes of pesticidal action. See U.S. Publication No. 20080226753.

One strategy for combating the development of resistance is to select a recombinant corn event which expresses high levels of the insecticidal protein. Another strategy would be to combine a second CRW specific insecticidal protein in the form of a recombinant event in the same plant or in an adjacent plant, for example, another Cry protein or alternatively another insecticidal protein such as a recombinant acyl lipid hydrolase or insecticidal variant thereof. See, e.g., WO 01/49834. Preferably, the second toxin or toxin complex would have a different mode of action than the first toxin, and preferably, if receptors were involved in the toxicity of the insect to the recombinant protein, the receptors for each of the two or more insecticidal proteins in the same plant or an adjacent plant would be different so that if a change of function of a receptor or a loss of function of a receptor developed as the cause of resistance to the particular insecticidal protein, then it should not and likely would not affect the insecticidal activity of the remaining toxin which would be shown to bind to a receptor different from the receptor causing the loss of function of one of the two insecticidal proteins cloned into a plant. Accordingly, the first one or more transgenes and the second one or more transgenes are preferably insecticidal to the same target insect and bind without competition to different binding sites in the gut membranes of the target insect.

Still another strategy would combine a chemical pesticide with a pesticidal protein expressed in a transgenic plant. This could conceivably take the form of a chemical seed treatment of a recombinant seed which would allow for the dispersal into a zone around the root of a pesticidally controlling amount of a chemical pesticide which would protect root tissues from target pest infestation so long as the chemical persisted or the root tissue remained within the zone of pesticide dispersed into the soil.

Another alternative to the conventional forms of pesticide application is the treatment of plant seeds with pesticides. The use of fungicides or nematicides to protect seeds, young roots, and shoots from attack after planting and sprouting, and the use of low levels of insecticides for the protection of, for example, corn seed from wireworm, has been used for some time. Seed treatment with pesticides has the advantage of providing for the protection of the seeds, while minimizing the amount of pesticide required and limiting the amount of contact with the pesticide and the number of different field applications necessary to attain control of the pests in the field.

Other examples of the control of pests by applying insecticides directly to plant seed are provided in, for example, U.S. Pat. No. 5,696,144. In addition, U.S. Pat. No. 5,876,739 to Turnblad et al. and its parent, U.S. Pat. No. 5,849,320, disclose a method for controlling soil-borne insects which involves treating seeds with a coating containing one or more polymeric binders and an insecticide. This reference provides a list of insecticides that it identifies as candidates for use in this coating and also names a number of potential target insects.

Although recent developments in genetic engineering of plants have improved the ability to protect plants from pests without using chemical pesticides, and while such techniques have reduced the harmful effects of pesticides on the environment, numerous problems remain that limit the successful application of these methods under actual field conditions. One such problem is the threat insect resistance poses to the future use of Bt plant-incorporated protectants and Bt technology as a whole. Specific IRM strategies, such as the high dose/structured refuge strategy, mitigate insect resistance to specific Bt proteins produced in corn, cotton, and potatoes. However, such strategies need monitoring of pest populations in order to quickly detect possible pest resistance.

Monitoring is also necessitated because from a farmer/producer's perspective, it is highly desirable to have as small a refuge as possible; thus some farmers choose to eschew the refuge requirements, and others do not follow the size and/or placement requirements. These non-compliance issues result in either no refuge or less effective refuge, and a corresponding increased risk of the development of resistance pests.

Due to potential compliance problems, regulatory requirements, and also the importance of trait durability, strategies to insure successful management and monitoring have been developed. These are implemented through field reports of unexpected damage by the target pest and population testing and sampling. Field monitoring data is used to determine normal level of pest damage in a field from a pest such as rootworm. Once field guidelines are established, reports of pest damage are evaluated to determine if crop damage is higher than the expected level, i.e. unexpected level of damage, due to failure of the transgene encoding pesticidal activity or to some alternative factor. For example, current sampling to identify resistance to traits conferring resistance or tolerance to pests that damage maize roots includes choosing plants from a field exhibiting an unexpected level of damage, determining trait expression, rating roots and, if necessary, determining alternative causes of damage or lodging such as non-target pest insect species, weather, physical damage, planting errors and other factors. In cases where the unexpected level of damage cannot be accounted for by factors other than a potential resistant pest population, remedial actions to control the spread of resistance are implemented. These required local actions include additional measures to control the pest, such as pesticide applications and cultivation practices which are both costly and resource intensive for the grower. Ultimately, remedial action can result in loss of the use of pesticidal transgenic plants for control of the pest.

Most countries, including the United States, require extensive registration requirements when transgenic crops are used in order to minimize the development of resistant pests, and thereby extend the useful life of known biopesticides. According to the United States Environmental Protection Agency (EPA) and United States Department of Agriculture (USDA), the EPA and USDA generally support the following strategy to manage the development of pest resistance to Bt toxins expressed in crops. That is, a structured refuge/high dose strategy should be employed for susceptible pests within the current understanding of the technology. The presence of an effective structured refuge, in combination with a high dose expression level of the Bt toxin, has the potential to delay the development of resistance in pests.

Refuges are non-Bt host plants that are managed to provide sufficient susceptible adult insects to mate with potential BT-resistant adult insects to dilute the frequency of resistance genes. The 1998 SAP subpanel on Bt crop resistance management suggested that production of 500 susceptible adults in the refuge should be available for mating with every potentially resistant adult in a BT field (assuming a resistance allele frequency of 5×10−2) (Final Report of the Subpanel on Bacillus thuringiensis (Bt) Plant-Pesticides and Resistance Management, February, 1998 (186 kb, PDF). Several strategies have been discussed (Fischoff 1992, McGaughey and Whalon 21992, Mallet and Porter 1992, Caprio 1994), but the current strategy recommends the use of plants with high dose expression and the provision of an external refuge in close proximity to the transgenic plants (ILSI HESI 1998). One of the most common examples of a refuge is where in a given crop, 80% of the seed planted may contain a transgenic event which kills a target pest (such as CRW), but 20% of the seed must not contain that transgenic event. The goal of such a refuge strategy is prevent the target pests from developing resistance to the particular biopesticide produced by the transgenic crop. Because those target insects that reach maturity in the 80% transgenic area will likely be resistant to the biopesticide used there, the refuge permits adult CRW insects to develop that are not resistant to the biopesticide used in the transgenic seeds. As a result, the non-resistant insects breed with the resistant insects, and, because the resistance gene is typically recessive, eliminate much of the resistance in the next generation of insects.

The problem with the refuge strategy is that in order to produce susceptible insects, some of the crop planted must be susceptible to the pest, thereby reducing yield. Thus, from a farmer's perspective, it is highly desirable to have as small a refuge as possible in order to maximize yields and profits. As a result, some farmers choose to eschew the refuge requirements, and other do not follow the size and/or placement requirements. These non-compliance issues result in either no refuge or less effective refuge, and a corresponding increased risk of the development of resistance insect pests. As a result, it is vital that accurate methods be developed in order to insure successful management of the pest and to promote durability of a resistance trait in maize and other plants.

### EXAMPLES

Embodiments of this invention can be better understood by reference to the following example. The foregoing and following description of the present invention is not intended to limit the claims, but rather are illustrative thereof. Therefore, it will be understood that the claims are not limited to the specific details of this example. It will be appreciated by those skilled in the art that other embodiments of the invention may be practiced without departing from the spirit and the scope of the disclosure, as defined by the appended claims. The disclosure of the reference set herein is incorporated herein by reference of its entirety for all purposes.

In the current invention, corn rootworm populations were sampled in problem fields and in control fields found within Iowa, USA. Problem fields were defined as fields with severe feeding injury to Bt maize by corn rootworm and were identified by farmers that contacted the extension service of Iowa State University. Problem fields contained plants that were goosenecked (bent at the plant-soil interface) and lodged (tilted in a pronounced manner), which are characteristic of feeding by corn rootworm larvae. Additionally, farmers noted a high abundance of rootworm adults in problem fields. Upon receiving notification of a problem field, the field was visited and the western corn rootworm present in the field was sampled. In all four problem fields, the vast majority of adult Diabrotica spp. present in the field were western corn rootworm. In one case a problem field was present on an Iowa State University research farm. With the exception of this Iowa State University research farm, maize roots were dug from the problem fields to evaluate rootworm feeding injury and the presence of Bt toxin was confirmed by ELISA with a kit (Envirologix, Portland, Me.). Roots were not sampled at random but were selected to confirm the presence of rootworm feeding.

Control fields were defined as fields not associated with unexpected feeding by corn rootworm on Bt maize. To allow for comparison with problem fields, only western corn rootworm were sampled from control fields. Five control fields, widely distributed throughout Iowa, were sampled. Three of the control fields were located on Iowa State University research farms. One control field was identified based on a grower complaint of heavy rootworm injury to non-Bt corn. Another control field was identified as part of a survey of corn rootworm abundance in Iowa (M. Dunbar pers. obs.). This field was the only control field with a history of Bt maize but there was not apparent rootworm feeding as evidenced by an absence of lodging by maize plants. Maize roots were not examined in control fields, so the extent of rootworm feeding is unknown.

Farmers and farm managers were interviewed to determine the crop history of the fields. Individuals were asked if Bt maize had been grown in the field, during which years, and what type of Bt maize (e.g., Cry3Bb1 or Cry34/35Ab1). No questions were asked about planting of refuge, size of refuge, or proximity of the refuge to the Bt field. For years in which Bt maize was not grown in a field, individuals were asked about the type of crop that was grown (e.g., maize or soybeans).

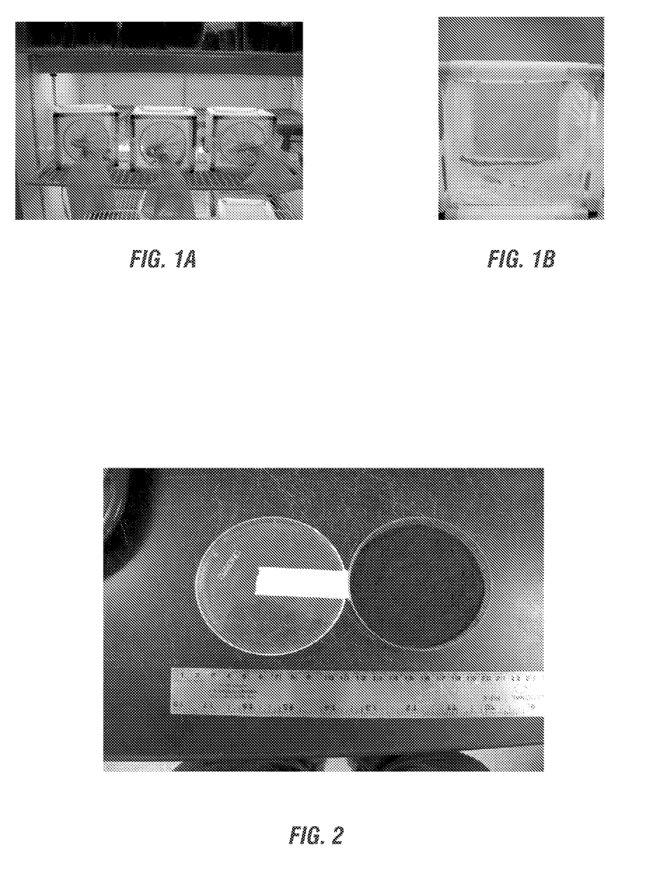

Adult western corn rootworm collected in the field were brought to Iowa State University where they were held in small cages (18 cm×18 cm×18 cm L×W×H) (Megaview Science, Taichung, Taiwan) and provided with food consisting of corn leaf tissue and an artificial diet (western corn rootworm diet, Bio-Serv, Frenchtown, N.J.). See FIGS. 1A, 1B. The water source for the adult beetles was 1.5% agar solid, which was 98.5% water by mass, and provided water to the adult western corn rootworm when consumed. Cages were held in an incubator (25° C.; 16/8 L/D) and individuals from each population were housed in separate cages. Adults were provided with an oviposition substrate that consisted of moist, finely sieved soil (<180 μm) placed in a 10 cm Petri dish. Eggs obtained from each population were placed separately in 45 mL plastic cups containing moistened sieved soil, and then sealed in a plastic bag and placed in a cold room at 8° C. for at least 5 months to break diapause. Following exposure to cold, eggs were stored for one week at 25° C. Eggs were washed from the soil using a screen with 250 μm openings and then placed atop moistened sieved soil held in a 10 cm Petri dish. See FIG. 2. Neonate larvae began hatching approximately one week thereafter.

Neonate larvae from each population were evaluated in laboratory bioassays for their survival on two transgenic maize hybrids, each of which contained a unique Bt toxin targeting com rootworm. One hybrid (DeKalb DKC 6169) produced Cry3Bb1. The other hybrid (Mycogen 2T789) produced Cry34/35Ab1. For both of these hybrids, we also evaluated rootworm survival on a near isogenic hybrid that lacked a gene for a rootworm active Bt toxin but otherwise was genetically similar to its respective Bt hybrid. In the case of Cry3Bb1 maize, the non-Bt hybrid was DKC 6172 (DeKalb) and for Cry34/35Ab1 maize the non-Bt hybrid was 2T777 (Mycogen).

Maize plants used in bioassays were grown in a greenhouse (25° C., 16/8 L/D) in 1 L containers made of clear plastic (Reynolds Food Packaging, Shepherdsville, Ky.) with supplemental lighting provided with 400 W high-pressure sodium bulbs (Ruud Lighting Inc., Racine, Wis.). Containers were filled with 750 mL of a 1:1 ratio of Sunshine Sun Gro SB300 and Sunshine Sun Gro LC1 potting soils (Sun Gro Horticulture Canada Ltd., Vancouver, British Columbia). Seeds were planted one per container at a depth of ca. 4 cm. Beginning two weeks after planting, plants were fertilized weekly with 100 mL of Peters Excel 15-5-15 Cal-Mag Special (Everris International, Geldermalsen, The Netherlands) at a concentration of 4 mg per mL.

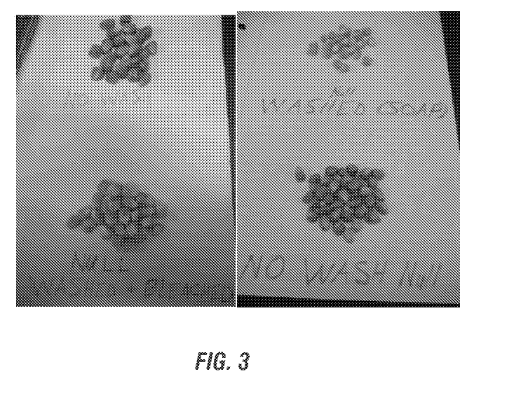

Maize seeds of 2T789 and 2T777 were coated with a seed treatment (CruiserMaxx 250, Syngenta, Basel, Switzerland), which contained the neonicotinoid insecticide Thiamethoxam. Prior to planting, this seed treatment was removed by washing ca. 50 seeds in a solution of 1 mL dish detergent (Ultra Palmolive Original, Colgate-Palmolive Company, New York, N.Y.) and 250 mL deionized water. Seeds were placed in the detergent solution for 20 minutes and agitated gently using a stirring plate and magnetic stirring bar. This process was repeated three times with seeds rinsed four times with deionized water between each time they were washed. Seeds were then rinsed four times and allowed to dry for approximately 12 hours, followed by one hour of soaking in a 10% bleach solution, during which they were stirred every 15 minutes. After seeds were removed from the bleach solution, they were rinsed 10 times with deionized water and then allowed to dry for at least 24 hours. This process removed virtually all visible signs of the seed treatment. See FIG. 3. Insecticidal seed treatment was not applied to DKC 6169 and DKC 6172. However, to ensure that no residual insecticide was present, seeds were bleached following the methods used with 2T789 and 2T777.

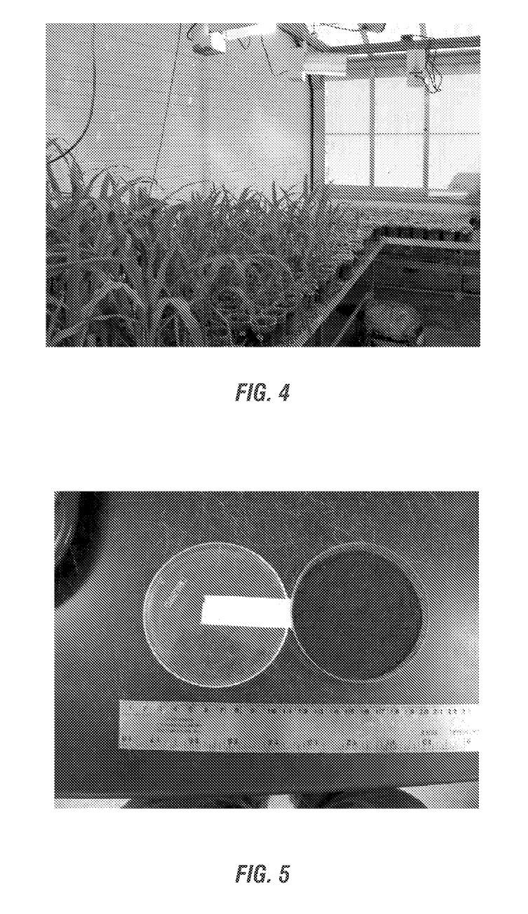

Plants were grown in a greenhouse for three to four weeks, until they contained at least five fully formed leaves (V5 stage), and then moved to incubators for bioassays. See FIG. 4. For bioassays, plants were first trimmed to a height of 20 cm to allow for storage in incubators. Two to three leaves were left on each plant but were trimmed to 8 cm long. See FIG. 6. Recently hatched larvae (less than 24 hours old) were removed from the soil's surface within their Petri dish using a fine brush and placed at the base of a maize plant on a root that had been exposed by moving away a small amount of soil. See FIG. 5. Maize plants remained in their original 1 L containers throughout the bioassay. Between 10 and 20 neonates were placed on the base of each plant. Larvae were distributed equally between Bt and nonBt maize plants. Cups containing plants and larvae were placed in an incubator for 17 days (25° C., 65% RH, 16/8 L/D), and plants were watered as needed.

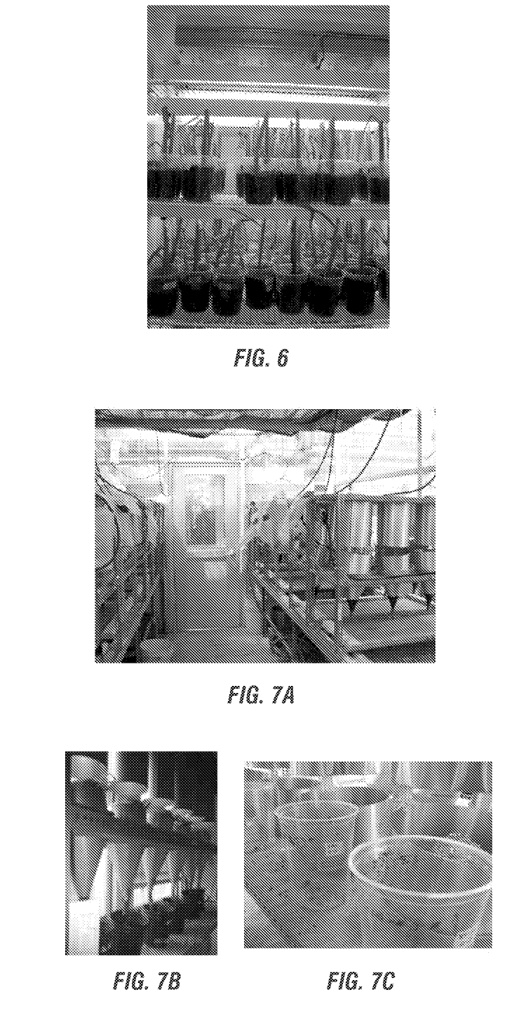

After 17 days, the aboveground biomass of the plant was excised and the soil, containing roots and larvae, was removed from the 1 L plastic container and placed on a Berlese funnel to extract larvae from the soil. See FIGS. 7A, 7B, 7C. A length of 17 days was selected for bioassays because it allowed sufficient time for some of the fastest developing larvae to reach the third and final instar. Root masses were held on Berlese funnels over 4 days and rootworm larvae were collected in 15 mL glass vials containing 10 mL of 85% ethanol. The average sample sizes per population were 12.7±(mean±standard deviation) bioassay cups for Cry3Bb1 maize and for its non-Bt counterpart, and 12.8±4.8 bioassay cups for Cry34/35Ab1 maize and for its non-Bt counterpart. There was insufficient western corn rootworm eggs to test one of the populations on Cry34/35Ab1 maize.

Data on the number of field-years (i.e., planting of one field for single year) during which problem fields and control fields were planted to Cry3Bb1 maize were compared using a G test of independence with a Williams's correction. See FIG. 8.

For each bioassay cup, proportional survival was calculated as the quotient of the number of larvae recovered after 17 days divided by the number of neonates initially placed in a bioassay container. The mean proportional survival for each population on each type of maize was analyzed with a two-way, mixed-model analysis of variance (ANOVA) (PROC MIXED in SAS). Data for the two types of Bt maize (Cry3Bb1 maize and Cry34/35Ab1 maize) were analyzed separately. The ANOVA included the fixed factors of field type (problem field vs. control field), maize hybrid (Bt maize vs. non-Bt maize) and their interaction. Random factors in the analysis were population, which was nested within field type, and the interaction between maize hybrid and population nested within field type. Survival data were transformed by the arcsine of the square root to ensure homogeneity of variance and normality of the residuals. Pairwise contrasts were conducted using the PDIFF option in PROC MIXED.

For each population, corrected survival was calculated as the complement of corrected mortality. Corrected mortality was determined using the correction of Abbott, and was calculated for each population by adjusting mortality of larvae from each bioassay cup with Bt maize by the average mortality on the non-Bt near isogenic hybrid. Average corrected survival for each population was compared between control fields and problem fields for Cry3Bb1 maize and for Cry34/35Ab1 maize based on a one-way ANOVA (PROC ANOVA in SAS).

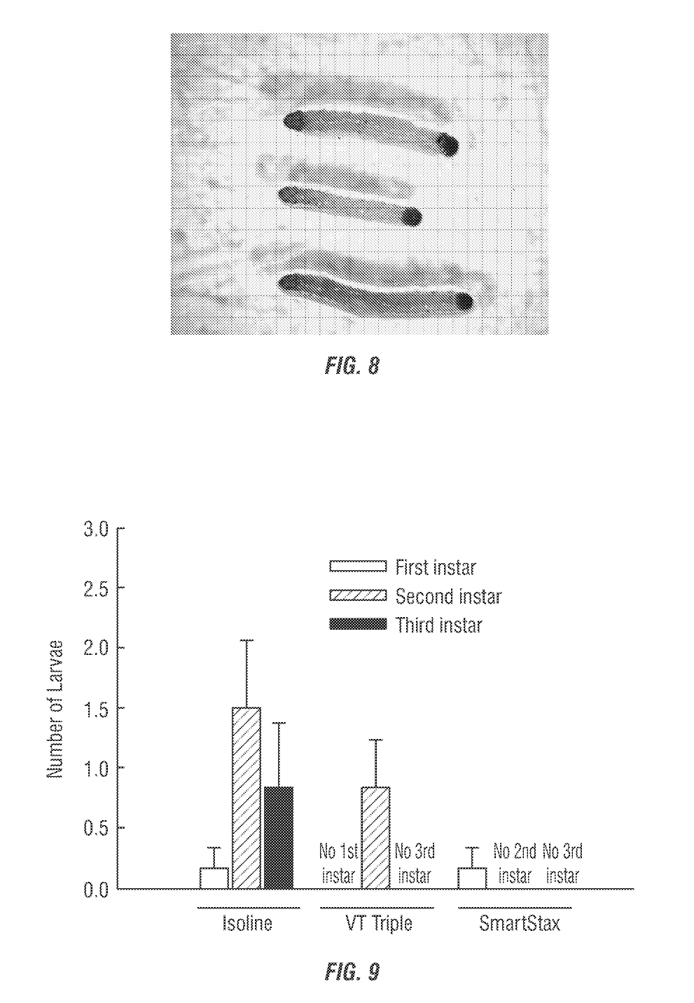

Corrected survival also was used to test the significance of three correlations among all populations sampled. The following correlations were tested: 1) corrected survival of populations on Cry3Bb1 maize and Cry34/35Ab1 maize, 2) corrected survival for populations on Cry3Bb1 maize and the number of years populations had been exposed to Cry3Bb1 maize in the field and 3) corrected survival for populations on Cry34/35Ab1 maize and the number of years populations had been exposed to Cry34/35Ab 1 maize in the field. Correlations were measured using a Pearson correlation coefficient and tested for significance against the null hypothesis of p=0 (PROC CORR in SAS). See FIG. 9.

What is claimed is:

1. A method for determining resistance of a pest to a pest resistant plant comprising the steps of:
obtaining a sample of pests suspected of having developed resistance to a pest resistant plant;
allowing the pests to reproduce;
harvesting the offspring of the pests;
growing a plurality of plants pest resistant plants;
placing the offspring of the pests onto the pest resistant plants;
removing the offspring of the pests from the pest resistant plants and soil after a sufficient time for incubation and feeding;
determining the offspring of the pests mortality rates; and
comparing the results to mortality rates of pests on non pest resistant plants.

2. A method according to claim 1, wherein the pests comprise insects selected from the orders Coleoptera, Diptera, Hymenoptera, Lepidoptera, Mallophaga, Homoptera, Hemiptera, Orthoptera, Thysanoptera, Dermaptera, Isoptera, Anoplura, Siphonaptera, and Trichoptera.

3. A method according to claim 2, wherein the pest is Diabrotica virgifera virgifera.

4. A method according to claim 1, wherein the pest resistant plant comprises plants selected from maize, sorghum, sunflower, soybean, wheat, alfalfa, rice, cotton, canola, barley, millet, potato and tomato.

5. A method according to claim 4, wherein the pest resistant crop plant is transgenic containing a toxin targeting the pest.

6. A method according to claim 5, wherein the toxin targeting the pest is a Bacillus thuringiensis toxin.

7. A method according to claim 5, wherein the pest resistant plant is Bacillus thuringiensis maize.

8. A method for determining resistance of a pest to a transgenic pest resistant plant comprising the steps of:
obtaining a sample of pests;
allowing the pests to reproduce and lay eggs;
harvesting the eggs of the pests;
allowing a sufficient time for the eggs to incubate;
collecting emerging larvae from the eggs of the pests;
growing a plurality of transgenic pest resistant plants;
placing the larvae collected from the eggs of the pests onto the transgenic pest resistant plants;
extracting the larvae from the pest resistant plants after a sufficient time for incubation and feeding;
determining larvae mortality rates by counting the larvae and measuring the instar based on head capsule width to calculate the resistance of the pests; and
comparing the results to the mortality rates of pests on non-transgenic pest resistant plants.

9. A method according to claim 8, wherein the pests comprise insects selected from the orders Coleoptera, Diptera, Hymenoptera, Lepidoptera, Mallophaga, Homoptera, Hemiptera, Orthoptera, Thysanoptera, Dermaptera, Isoptera, Anoplura, Siphonaptera, and Trichoptera.

10. A method according to claim 9, wherein the pest is Diabrotica virgifera virgifera.

11. A method according to claim 8, wherein the transgenic pest resistant plant comprises plants selected from maize, sorghum, sunflower, soybean, wheat, alfalfa, rice, cotton, canola, barley, millet, potato and tomato.

12. A method according to claim 11, wherein the transgenic pest resistant plant contains a toxin targeting the pest.

13. A method according to claim 12, wherein the toxin targeting the pest is a Bacillus thuringiensis toxin.

14. A method according to claim 11, wherein the transgenic pest resistant crop plant is Bacillus thuringiensis maize.

15. A method according to claim 8, wherein the sample of pests are placed into cages.

16. A method according to claim 15, wherein the cages are held in an incubator at 25° C.

17. A method according to claim 8, wherein the sample of pests are provided with food comprising corn leaf tissue and an artificial diet.

18. A method according to claim 17, where the artificial diet comprises western corn rootworm diet.

19. A method according to claim 18, wherein the water comprises 1.5% agar solid, which is 98.5% water by mass.

20. A method according to claim 19, wherein the eggs and soil are sealed in a plastic bag and placed in a cold room at 8° C. for at least 5 months.

21. A method according to claim 20, wherein after exposure to the cold room at 8° C. the plastic cups containing the eggs and sieved soil are further stored for approximately one week at 25° C.

22. A method according to claim 21, wherein the eggs after storage for approximately one week are washed out from the sieved soil in the plastic cups using a screen with 250 μm openings.

23. A method according to claim 8, wherein the larvae of the sample of pests are placed upon the exposed roots of the plurality of transgenic pest resistant plants.

24. A method according to claim 8, wherein the plurality of transgenic pest resistant plants are composed of different hybrids.

25. A method according to claim 8, wherein the plurality of transgenic pest resistant plants are grown from seeds.

26. A method according to claim 25, wherein the seeds are washed to remove any seed treatment.

27. A method according to claim 26, wherein the seed treatment comprises an insecticide, a pesticide, or a fungicide.

28. A method according to claim 27, wherein the seed treatment is removed by washing with a detergent solution, a bleach solution, and water.

29. A method according to claim 28, wherein the containers are filled with soil that comprises a 1:1 ratio of Sunshine Sun Gro SB300 and Sunshine Sun Gro LC1 soils.

30. A method according to claim 8, wherein 10-20 larvae are placed on the exposed roots of each transgenic pest resistant plant.

31. A method according to claim 8, wherein the sufficient time for incubation and feeding comprises approximately 17 days.

32. A method according to claim 8, wherein extracting the larvae from the pest resistant plants comprises a device for extracting soil insects and other micro fauna.

33. A method according to claim 32, wherein the device for extracting soil insects and other micro fauna is a Berlese funnel.

34. A method according to claim 8, wherein the sample of pests are obtained from a problem field with severe feeding injury.

35. A method selecting a type of hybrid to plant in a plot suspected of having pests that are resistant to pest resistant plants comprising:
performing the assay of claim 1;
selecting a type of pest resistant plant or strategy to employ based upon the results of said assay; and
planting the same.

36. A method according to claim 35, wherein said selecting of a type of plant resistant plant or pest resistant strategy includes one of the following:
a. gene stacking the transgenic pest resistant plant to include more than one pesticidal activity;
b. utilizing a different strain of transgenics to optimize toxic effects;
c. selecting a recombinant event in the transgenic pest resistant plant which expresses high levels of pesticidal proteins;
d. combine a chemical pesticide with a pesticidal protein expressed in the transgenic pest resistant plant; or e. treating seeds of the transgenic pest resistant plants with forms of pesticide applications.
